(19) United States
(12) Patent Application Publication
(10) Pub. No.: US 2010/0086496 A1
(43) Pub. Date: Apr. 8, 2010

(54) ORAL CARE PACKAGE

(76) Inventor: Patrick Lafferty, Tarpin Springs, FL (US)

Correspondence Address: A PATENT LAWYER CORP, PLC;R WILLIAM GRAHAM, 22 S ST CLAIR ST, DAYTON, OH, 45402, US

(21) Appl. No.: 12/570,347

(22) Filed: Sep. 30, 2009

Related U.S. Application Data
(60) Provisional application No. 61/103,485, filed on Oct. 7, 2008.

(51) Int. Cl. A61K 9/68; A61P 1/02

(52) U.S. Cl. 424/48

(57) ABSTRACT

An oral care package includes a package wrapping a tooth pick (or preferably a combined pick and flossing device) impressed within or adjacent to a stick of chewing gum in such manner that the pick (or pick/floss device) may be easily removed from the package first and used to dislodge matter from between the teeth and along the gums of the user prior to chewing the gum.

This application claims the benefit of provisional application Ser. No. 61/103,485 filed Oct. 7, 2008.

### BACKGROUND OF THE INVENTION

1. Field of the Invention

This invention relates generally to oral hygiene appliances and processes and more particularly, but not by way of limitation, to an oral care package containing chewing gum, tooth pick and a dental floss tool housed within a package which together provides a suitable utility for cleaning the occlusions on top of the teeth and between the gums when one does not have access to typical oral care products.

2. Prior Art

In the case where one does not have access to oral care products such as a toothbrush and toothpaste, dental floss, and mouth wash to permit complete oral hygiene, there is often a need to clean one's teeth and freshen one's breath. Previously, toothpicks have been routinely used for removal of food particles lodged between teeth. Toothpicks are typically used after meals to remove food particles but are not typically carried on one's person.

Also, there exist a number of flossing devices which aid in removal of food from one's teeth and massage and toughen the gums. Chewing gum is also known to aid in teeth cleaning and oral hygiene through mastication by removing particles of food left in irregular surfaces and occlusions of the upper most surfaces of the teeth. While these utilities exist in the marketplace, there is a need to improve the art.

### SUMMARY OF THE INVENTION

An object of the invention is to provide an oral care package which includes a combined chewing gum, tooth pick and flossing device in a unique package.

A further object is to provide a package wrapping a tooth pick (or combined pick and flossing device) impressed within a stick of gum in such manner that the pick/floss may be easily removed from the package first and used to dislodge matter from between the teeth of the user prior to chewing the gum.

Another object is to provide various mechanisms for packaging the combined elements to meet the purposes and advantages of the present invention.

Accordingly, one embodiment of the invention is directed to an oral care kit. The package includes a package wrapping a tooth pick (or preferably a combined pick and flossing device) impressed within or adjacent to a stick of gum in such manner that the pick (or pick/floss device) may be easily removed from the package first and used to dislodge matter from between the teeth of the user and to massage the gums prior to chewing the gum which may contain additional anti-carries chemicals or compounds.

Other and further objects and advantages will either be understood or will be pointed out hereinafter.

### BRIEF DESCRIPTION OF THE DRAWINGS

FIG. 1 shows a perspective view a preferred embodiment of the present invention with components thereof adjacent thereto;

FIG. 2A shows a plan view of a dental tool impressed into a stick of gum;

FIG. 2B shows a side sectional view of 2A;

FIG. 2C shows a plan view of another dental tool impressed into a stick of gum;

FIG. 2D shows a side sectional view of 2C;

FIG. 3A shows a wrapped stick of gum and dental tool;

FIG. 3B shows the dental tool exposed in a partially opened wrapper;

FIG. 3C shows the dental tool removed from the stick of gum with the impression in the gum exposed in the open wrapper;

FIG. 3D shows the stick of gum removed from the wrapper;

FIG. 4A shows another wrapped stick of gum having a dental tool therein;

FIG. 4B shows the stick of gum and dental tool partially exposed in a partially opened wrapper;

FIG. 4C shows the dental tool and the stick of gum removed from a wrapper configuration;

FIG. 4D shows the dental tool and the stick of gum in another wrapper configuration;

FIG. 5A shows another configuration of a wrapped stick of gum having a dental tool therein;

FIG. 5B shows the stick of gum and dental tool exposed in a partially opened wrapper disposed in a tray;

FIG. 5C shows the dental tool removed from the stick of gum with the impression in the gum exposed in the open wrapper;

FIG. 5D shows the stick of gum being removed from the wrapper; and

FIG. 6 shows a perspective view another preferred embodiment of the present invention with components thereof.

### DETAILED DESCRIPTION OF THE INVENTION

Referring now to the drawings, the oral care package of the present invention is generally designated by the numeral 10. Like numerals indicate corresponding parts in the various views of the components described herein. The components generally include a package 11 having one or more wrappers 12, each of which includes paper or foil covering 14, a stick of gum 16 and the dental tool 17.

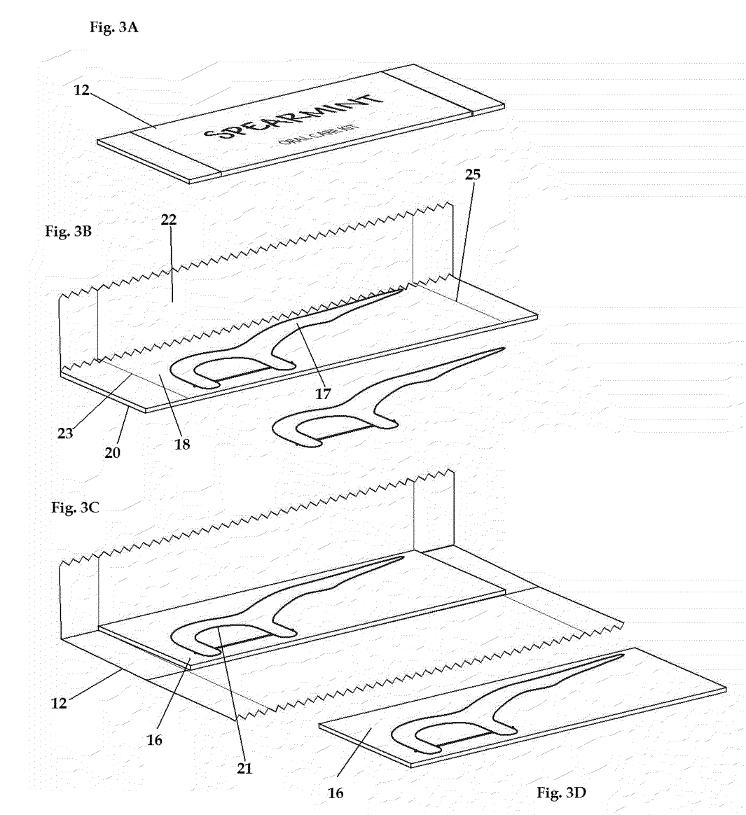

The packaging wrappers 12 can include a number of components as set forth herein. As seen in FIG. 3B, the wrapper 12 can include a conventional multi-fold wrap 14A which can be made of a waxed-paper or foil or other suitable material. Here, the stick of gum 16 can be disposed between a first fold 18 and a second fold 20 with the dental tool 17 depressed into the first fold and thereby causing an impression 21 in gum 16. A third fold 22 can be disposed over the dental tool 17 to enclose about the gum 16 and dental tool 17 once ends of the folds 18, 20 and 22 are folded over along fold lines 23 and 25. Once enfolded, the tool 17 and gum 16 are aided from becoming contaminated.

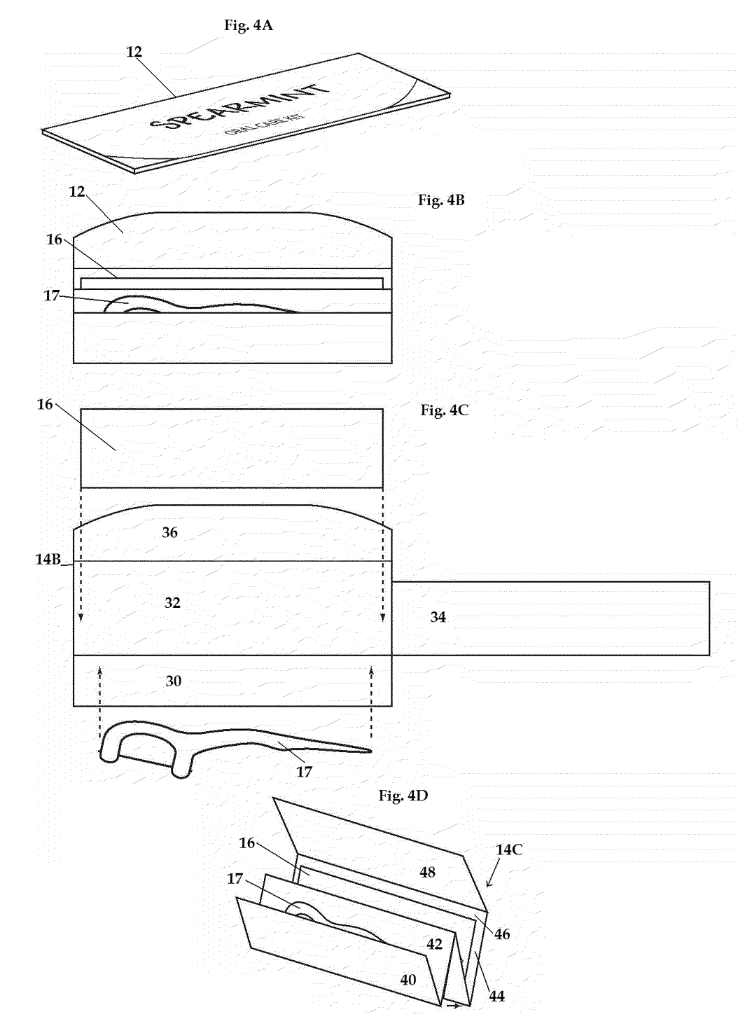

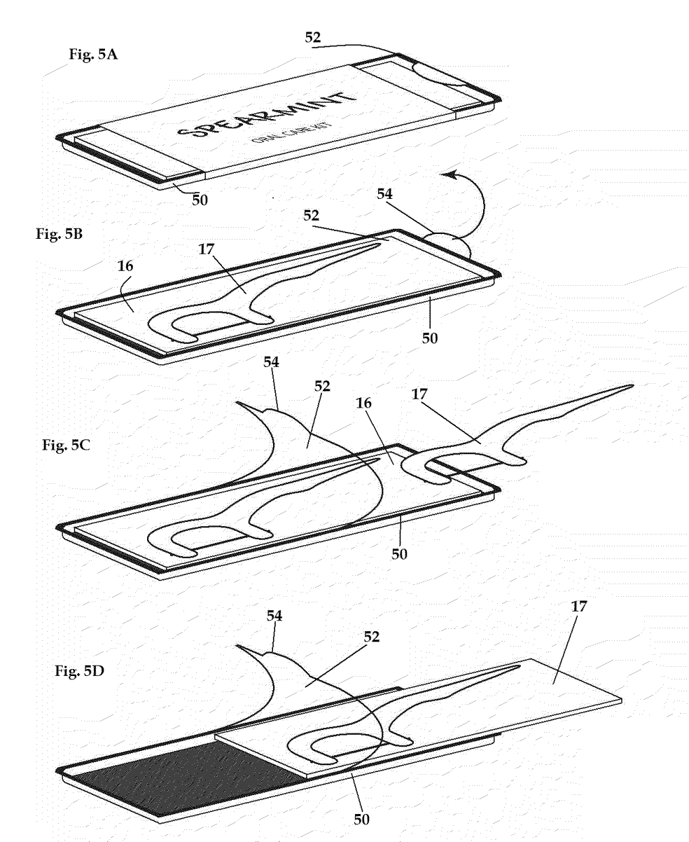

FIGS. 4B-C depict another multi-fold wrap 14B having folds 30, 32, 34, and 36 which separate gum 16 (with fold folds 32 and 34 containing gum 16) and tool 17. FIG. 14C depicts another multi-fold wrap 14C which shows accordion like folds 40, 42, 44, 46, and 48 which separately contain the gum 16 and tool 17. Still another version of the wrapper 12 can be seen in FIGS. 5A-D wherein the gum 16 includes tool 17 depressed therein and can be contained within a tray 50 having a biodegradable film 52 sealed to an edge of the tray 50 to contain the gum 16 and tool 17. The film 52 can be formed with a tab 54 to aid in removal from tray 50.

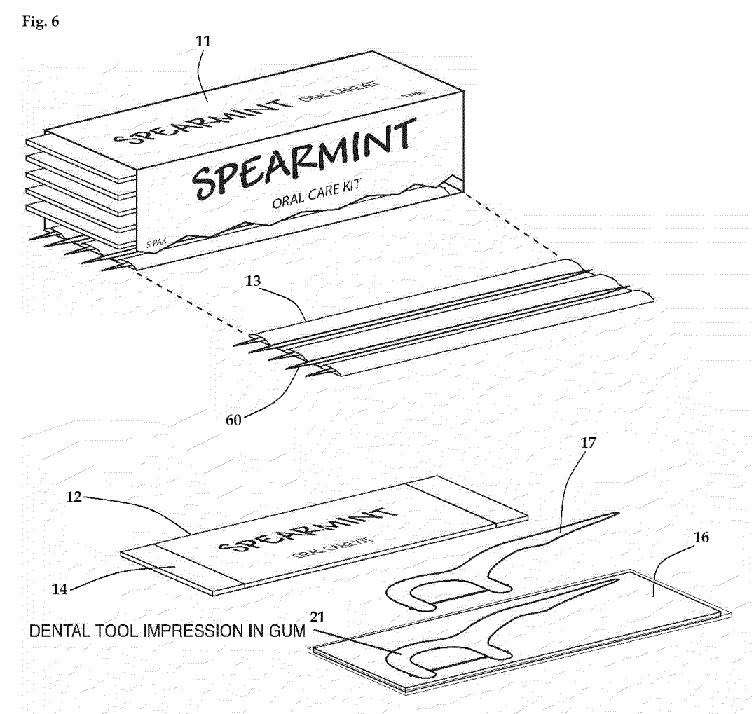

Another embodiment of the invention is seen in FIG. 6 wherein the package 11 can also include a corrugated insert wrapper 13 which has a plurality of longitudinal spaces defined therein to receive or contain one or more conventional wood, plastic, or composition toothpicks 60.

The gum itself 10 can preferably be any of suitable kind or types of chewing gum of which preferably can be naturally or sugar free sweetened or un-sweetened and can preferably contain one or more anti-carries additive(s).

It is also contemplated that there are many gums on the market which may be effective tooth cleaning properties. The gum 16 can be provided with appropriate ingredients to enable cleaning action on irregular surfaces of teeth and/or attract into itself and entrap food material from the teeth whether it be material on the teeth or between the teeth and whether it be material in the nature of a film of material or whether it be material in the nature of particles of food or the like.

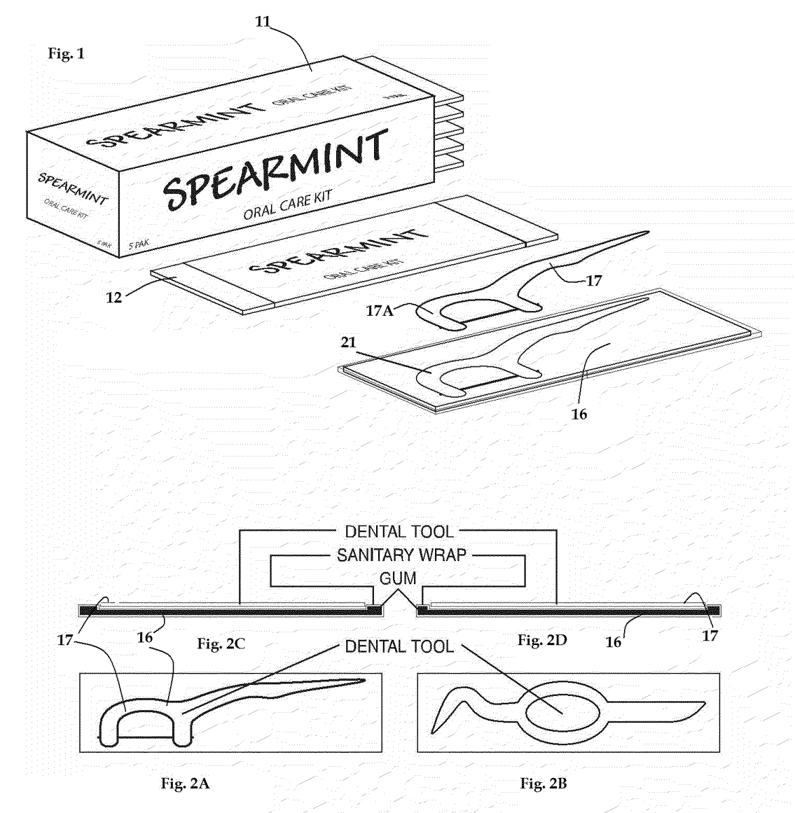

The dental tool 17 can be of any suitable configuration provided it is a length to permit it to be easily accessed and/or disposed in the wrapper 12 and or gum 16 as the embodiments contemplate. The tool 17 can preferably include a flossing end 17A as seen in many of the figures. However, a conventional toothpick can be employed in lieu of tool 17. FIG. 2C depicts an alternative tool 17 configuration.

In operation, the user extracts the tool 17 for use in removing any food from one's teeth. Then, a piece of chewing gum 16 can also then be removed from the wrapper 12 to further aid in cleaning and oral hygiene including the freshening of breath. The use of the invention is well suited for use after meals when there is most likely to be foreign matter such as food particles on or between the teeth in addition to any film of material which generally forms as a coating on the teeth at frequent intervals and contribute to bad breath.

According to the present invention, this method of use provides that the teeth and oral cavity will be beneficially affected with the result that decay will be greatly reduced and the teeth will be left in cleaner and healthier condition. It is apparent that the above invention is readily subject to modifications, derivations, and improvements and is intended as illustrative and the claims to the invention should be entitled to any of the same.

What is claimed is:

1. An oral care kit, which includes:
a package containing at least one piece of chewing gum and a dental tool wrapped within said package in such manner that said dental tool can be easily removed from said package and used to perform a hygienic operation in one's mouth prior to removing and chewing said gum.

2. The oral care kit of claim 1, wherein said gum contains anti-carries substance.

3. The oral care kit of claim 1, wherein said dental tool includes one of a tooth pick and a flossing device wrapped within said package in such manner that said dental tool may be easily removed from said package and used to either dislodge matter from between the teeth dislodge matter from between the teeth or massage gums prior to chewing said gum.

4. The oral care kit of claim 1, wherein said dental tool is disposed adjacent said gum.

5. The oral care kit of claim 4, wherein dental tool pick is impressed within said gum.

6. The oral care kit of claim 3, wherein said tooth pick and flossing device are integrally formed.

7. The oral care kit of claim 1, wherein said package includes a multi-fold wrapper having a first portion for receiving said gum thereagainst and a second portion for enclosing about said gum wherein said wrapper substantially encloses said gum.

8. The oral care kit of claim 7, wherein said wrapper substantially encloses said gum and said dental tool.

9. The oral care kit of claim 7, wherein said gum is disposed between said first and second portions and to substantially enclose said gum, and said dental tool is disposed outside of and adjacent to said wrapper.

10. The oral care kit of claim 9, wherein said wrapper includes a third portion enclosing said dental tool.

11. The oral care kit of claim 10, wherein said dental tool includes one of a tooth pick and a flossing device wrapped within said package in such manner that said dental tool may be easily removed from said package and used to either dislodge matter from between the teeth dislodge matter from between the teeth or massage gums prior to chewing said gum.

12. The oral care kit of claim 1, wherein said dental tool is readily removable prior to said gum.

13. The oral care kit of claim 7, wherein said wrapper is biodegradable.
